(19) United States
(12) Patent Application Publication
(10) Pub. No.: US 2017/0326086 A1
(43) Pub. Date: Nov. 16, 2017

(54) USE OF HEMOGLOBIN EFFECTORS TO INCREASE THE BIOAVAILABILITY OF THERAPEUTIC GASES

(71) Applicants: Martin SAFO, Richmond, VA (US); Kevin R. WARD, Glen Allen, VA (US)

(72) Inventors: Martin SAFO, Richmond, VA (US); Kevin R. WARD, Glen Allen, VA (US)

(73) Assignee: VIRGINIA COMMONWEALTH UNIVERSITY, Richmond, VA (US)

(21) Appl. No.: 15/629,302

(22) Filed: Jun. 21, 2017

Related U.S. Application Data
(63) Continuation of application No. 13/990,252, filed on May 29, 2013, now abandoned.
(60) Provisional application No. 61/418,503, filed on Dec. 1, 2010.

(51) Int. Cl. A61K 31/196; A61K 33/16; A61K 33/00; A61K 31/665; A61K 31/6615; A61K 31/192; A61K 31/02; A61K 38/42; A61K 9/00

(52) U.S. Cl. CPC A61K 31/196; A61K 38/42; A61K 33/16; A61K 33/00; A61K 9/0019; A61K 31/6615; A61K 31/192; A61K 31/02; A61K 31/665

(57) ABSTRACT

Methods which increase the bioavailability of beneficial gases in the circulatory system are provided. The methods involve administering agents that changes the binding affinity of a medicinal gas such as NO, CO, H₂S, N₂O, SO, SO₂ and O₂ for IIb and/or hemoglobin based oxygen carriers (HBOCs). The change results in increased release of gases carried by Hb and HBOCs. As a result, the concentration of the gases in circulation is raised, and they are more available to exert their beneficial effects, e.g. in the treatment of disease or conditions caused by low levels of the gases. The methods are optionally used together with administration of medicinal gases and/or administration of HBOCs and/or other non-HBOC gas carriers such as PFC, and as (or in conjunction with) diagnostic methods.

### BACKGROUND OF THE INVENTION

### Field of the Invention

The invention generally relates to methods which increase the bioavailability of therapeutic gases that bind hemoglobin (Hb). In particular, the methods involve the administration of agents that change the binding of medicinal gases to hemoglobin (Hb) and hemoglobin based oxygen carriers (HBOCs), in order to promote release of gases carried by Hb and HBOCs, e.g. oxygen (O₂), nitric oxide (NO), carbon monoxide (CO), hydrogen sulfide (H₂S), etc.

### Background of the Invention

Hemoglobin (Hb) exists in equilibrium between two alternative states, the tense or T state (unliganded or deoxygenated Hb), which possesses low oxygen affinity, and the relaxed or R state (liganded or oxygenated Hb), which has a high oxygen affinity. The tetrameric Hb structures are composed of two αβ dimers; arranged around a 2-fold axis of symmetry, resulting in a large central water cavity in the structures. The allosteric equilibrium of Hb can be modulated by effectors. A shift toward the relaxed state left-shifts the oxygen equilibrium curve (OEC), producing a high affinity Hb that more readily binds and holds oxygen. A shift toward the T state (right-shift) produces a low affinity Hb that readily releases oxygen. For instance, preferential binding of the endogenous Hb allosteric effector, 2,3-diphosphoglycerate (2,3-DPG) at the central water cavity in the T state leads to additional stabilization of the T state form relative to the R state, further decreasing the affinity of T state Hb for oxygen, causing release of more O₂ from the Hb, and thus increasing tissue oxygenation.¹ The degree of shift in the OEC is reported as an increase or decrease in P₅₀ (oxygen tension at 50% Hb O₂ saturation). The degree of allosteric character/cooperativity demonstrated by Hb is described by the Hill coefficient (n).

Since the discovery of 2,3-DPG, there have been ongoing efforts to develop Hb-based drugs to treat several diseases for which a transient increase in oxygen delivery to tissues is beneficial. ²⁻²¹ Since only 25% of hemoglobin-bound oxygen is released to tissues in each circulation cycle, a pharmacologically induced increase in P₅₀ translates into a significantly higher oxygen delivery, provided that the affinity remains high enough to allow oxygen saturation in the lungs. Three of such studied compounds are inositol hexaphosphate (IHp),^{18-20, 22, 23} the antilipidemic drug bezafibrate (BZF),^{2, 21} and urea derivatives of BZF.^{2, 3} Earlier studies have suggested exclusive binding of these effectors to deoxygenated Hb;^{4,5,7,24} however it's recently been shown that these compounds bind to both the T and R state conformations to modulate Hb function.^{22, 23, 25-27} The therapeutic applications of IHP for increasing tissue oxygen in ischemic related diseases was prevented by its limited absorption profile; ^{18, 19} while BZF showed significant binding to serum preventing its transport into RBC's.

Abraham and co-workers developed other potent right-shifting effectors with less binding to serum albumin. One of the compounds, Efaproxiral or RSR-13, (2-[4[[(3,5-dimethylanilino)carbon]methyl]phenoxyl]-2-methylpropionic acid) is a very potent synthetic allosteric effector of Hb, inducing the protein to exhibit low oxygen affinity and very low cooperativity by allosterically stabilizing the deoxygenated Hb.^{4, 5, 7, 24} Due to this property, RSR-13 has been studied to treat hypoxic- or ischemic-related diseases.^{9, 11-13, 15-17, 28-31} RSR-13 binds to Hb in a similar fashion as BZF or L35, leading to significant lowering of Hb affinity for oxygen.^{4, 5, 7, 24} Various structural modifications were made to RSR-13 by Abraham and co-workers to design more potent right-shift effectors.^{4, 6, 24, 32, 33} The compounds categorized into several classes differ mainly in the substitution pattern at the benzene ring, linker atoms, and the methylpropylene acid group. The substitutions have brought about subtle, but significant differences in the Hb binding modes that may explain the differences in their allosteric activities.

In early in vivo experiments, RSR-13 was shown to increase the release of oxygen to tissues,^{15, 16} and to induce hemodynamic changes associated with higher concentrations of circulating oxygen.^{13, 14} Phase I studies confirmed the safety of Efaproxiral and its capacity to increase the P₅₀ of whole blood by 10 Torr at doses of 75-100 mg/kg. RSR-13 has been investigated for several hypoxic- or ischemic-related diseases, including stroke and myocardial ischemia;¹⁷ globally hyperfused states such as hemorrhage and sepsis; and as a means to hyperoxygenate tumors making them more susceptible to radiation therapy.^{9, 11, 12} RSR-13 also underwent Phase III clinical trials as an adjunct to whole brain radiation therapy in the treatment of brain metastases.^{9, 11, 12}

Several studies have attempted to explain the mechanism of action of RSR-13, which suggest that RSR-13 perhaps act through binding of the compound to deoxygenated Hb to stabilize the T state conformation.^{4, 5, 7, 24} Our group has determined the crystal structure of deoxygenated Hb in complex with RSR-13, and shows a pair of RSR-13 molecules binding in a symmetry-related fashion in the central water cavity of deoxyhemoglobin,⁷ each molecule making several hydrogen-bond and hydrophobic interactions with three subunits (two α-subunits and one β-subunit of the protein) of the tetramer. The allosteric transition from T to R is characterized by movement of several central water cavity residues, and binding of RSR-13 to the T state conformation or deoxyhemoglobin restrains the movement of these residues, thus stabilizing the T state with concomitant decrease Hb oxygen affinity. Specifically, the propionate moiety of RSR-13 which is located in a pocket formed by the residues α1Pro95, α1Thr137, α1Arg141, β2Tyr35 and β2Trp37, makes a water-mediated hydrogen-bond interaction with the guanidinium group of α1Arg141, restraining this residue from transitioning to the R state position. The 3,5-dimethylbenzene moiety of RSR-13 also makes several hydrophobic interactions with the protein G helix, and restrains it from moving to its R state position. Additionally, there is a unique hydrogen-bond interaction between the RSR-13 carbonyl oxygen and the side-chain amino group of α1Lys99 which stabilizes the T state further. Arnone and co-workers have also identified several of these RSR-13 contact residues, including βTrp37, βTrp35, αArg141 and the G helix residues to be the major region of the quaternary constraint.^{34, 35}

It has also been suggested that direct interactions of allosteric effectors to or R state or liganded Hb can also reduce the Hb affinity for oxygen. The crystal structures of liganded R state Hb in complex with BZF and L35 have been determined, and show the compounds bound to the surface of the protein, close to the E helix of the α-heme.^{25, 27, 36} The mode of BZF and L35 binding is believed to increase steric hindrance at the distal pocket by αHis58, prompting the suggestion that the observed low-oxygen affinity of the liganded Hb in the presence of these effectors is partly due to steric hindrance at the distal cavity. Most likely, RSR-13 also binds to liganded Hb in a similar fashion as BZF or L35.³⁷ Thus, it is clear that RSR-13 and similar effectors are capable of binding to both liganded and deoxygenated Hb, and their regulatory effect on Hb function appears to result from their interactions with both deoxygenated Hb and ligated forms of Hb resulting in decreased Hb affinity for ligands.

RSR-13 has also been proposed as a means to treat carbon monoxide poisoning by off-loading CO from hemoglobin through the decrease in ligand affinity similar to that of off-loading oxygen. This is described in U.S. Pat. No. 5,525,630, where the use of RSR-13 to clear CO bound to Hb in rats exposed to sub-lethal, circulating COHb level of approximately 40% was shown.

Carbon monoxide (CO), nitric oxide (NO) and hydrogen sulfide (H₂S) are produced by many types of cells and serve as beneficial pleotrophic modulators in health and disease including acting as vasorelaxing and anti-inflammation factors.³⁸⁻⁴² For example, NO, H₂S, and CO are produced by the body in increasing quantities in states of injury as a means to assist in preserving microcirculatory blood flow and to modulate inflammation. As such, there has been great interest in developing the means to exogenously deliver NO, H₂S and CO for therapeutic purposes and/or to enhance their endogenous production. Strategies to enhance delivery of NO to tissues have included inhalation of NO, use of medications to increase NO concentrations or to increase production by the body, and mechanical methods of increasing shear stress at the vascular level to promote production of NO. The use of inhaled NO has been used to treat pulmonary hypertension and other diseases such as sepsis.⁴³⁻⁴⁹ However, there are limitations to dosing of exogenous NO. NO has a very short half-life and when it is present in plasma, it is believed to be rapidly sequestered by hemoglobin and inactivated and thus is not available to exert is beneficial effects. At high concentrations of NO, this results in hemoglobin being reduced to methemoglobin (metHb), which can be toxic at high doses. It is widely accepted that Hb-induced hypertension is primarily caused by NO either reacting with deoxygenated Hb to form nitrosyl Hb or by reacting with oxygenated Hb to form methemoglobin and nitrate.⁵⁰⁻⁵² Vasoconstriction due to the scavenging of NO by Hb is even more significant when Hb is present in blood vessels outside erythrocytes, such as during hemolysis.⁵³ Thus, provision of exogenous NO to produce favorable vasodilatory and anti-inflammatory effects is not simply a matter of increasing the concentration of administrations because of NO's interaction with hemoglobin.

It is also important to note that binding of NO to hemoglobin is of even bigger concern with the use of hemoglobin based oxygen carriers (HBOCs). Hemoglobin (Hb) functions by binding and transporting oxygen from the lungs to the tissues, and offloading to respiring cells. Due to several problems associated with blood transfusion, cell-free HBOCs have been under investigation for several decades for potential use to support blood oxygen transport during hemorrhage shock, sepsis, homolysis and various ischemic insults ranging from stroke to myocardial infarction, to traumatic brain and spinal cord injury, among others. HBOCs have thus been developed as a mean to deliver oxygen to tissues as an alternative to native human blood. In general, these agents are made by taking human or bovine hemoglobin out of red blood cells and processing it in a way that produces linked tetramers and other configurations of Hb. These can be further modified if desired through either reencapsulation in an artificial membrane or through processes such as PEGylation in order to increase the circulating half-life of the HBOC. Although, these blood substitutes have demonstrated efficacy in both animal models and humans, several serious safety problems, including death, have impeded their clinical use.^{54, 55} For example, a characteristic and persistent side effect of many of these HBOCs has been the propensity to cause vasoconstriction and increase blood pressure due to the scavenging of endogenously produced NO.⁵⁶ NO is, as described above, an important signaling molecule, and is produced naturally from L-arginine by nitric oxide synthases, primarily in the vascular smooth muscle and endothelium. NO binds to guanilate cyclase in smooth vessels to cause vasorelaxation.^{38, 57-60} In fact, Hb has also been suggested to have a secondary function as a nitrite reductase, converting nitrite ion to NO.⁶¹

A general side effect in the processing and production of HBOCs is that they become potent scavengers of NO⁶² and as a result can cause undesired increases in blood pressure during their use to treat hemorrhage, which may paradoxically result in more hemorrhage. This has resulted in part in no HBOC being approved by the FDA due to concerns that their use causes increased bleeding. Hemorrhage from many causes results in increased production of endogenous NO, as the body's way of attempting to lessen bleeding by relaxing blood vessels and maintaining microcirculatory blood flow and tissue oxygenation. Unfortunately, the potential benefits of administering HBOCs in order to treat hemorrhage (e.g. to replace lost blood and/or increase oxygen delivery to tissue), are nullified or at least lessened when the HBOCs scavenge NO, causing an increase in blood pressure and thus additional hemorrhage. In addition, this scavenging may reduce microvascular blood flow thus worsening tissue perfusion and oxygenation. Other complications have included a higher than expected incidence of myocardial damage which may be due to enhanced binding of NO by HBOCs.

Another potentially unwanted side effect of HBOCs is that the P₅₀ of the resulting HBOC is sometimes significantly reduced. This has the effect of decreasing the ability of the HBOC to release oxygen in the setting for which it is designed. While several manufacturers claim a low P₅₀ is an advantage, replacement of significant amounts of native hemoglobin with an HBOC of low P₅₀ ultimately results in tissue hypoxia. While the ability of an HBOC to tightly bind and carry large amounts of O₂ might appear to be advantageous, if the O₂ is not expeditiously released to the tissues during circulation, then the purpose of the increased O₂ binding capacity is defeated.

Over the last two decades it has been shown that, similar to NO, CO is endogenously produced in many types of cells (e.g. by hemeoxygenase) and has therapeutic value, serving as a microvascular relaxation factor and as an anti-inflammatory agent.³⁸⁻⁴² It has also been demonstrated in several studies that CO delivered exogenously, either through inhalation or via CO-releasing molecules (CORMs), or even as part of an HBOC, can also act as anti-inflammatory, vasodilator, and tissue protectant.⁶³⁻⁶⁶ However, there is a potential toxicity associated with exogenous delivery of CO, due to increase levels of carboxyhemoglobin (COHb), which translates into impaired oxygen delivery to tissues and organs.^{67, 68} This is especially true in cases of global hypoperfusion or if patients have severe coronary or cerebrovascular disease, where the resulting levels of COHb could be dangerous. For example, levels of exogenous CO needed to show clinical efficacy have resulted in COHb levels of 15-20%. Such levels, while possibly tolerated in young individuals with no co-morbidities, is unlikely to be well tolerated by the elderly or others who may also have serious underlying cardiovascular and cerebrovascular disease. This detrimental effect would be made worse in situations where supplemental oxygen is not available (e.g. on the battlefield). CO binds to Hb with an affinity 240 times higher than that of oxygen.

As is the case with NO, HBOCs also scavenge CO, requiring the use of large amount of CO when HBOC is used as a delivery vehicle, potentially leading to toxicity problem as described above. HBOC-induced vasoconstriction due to scavenging of endogenously produced CO is also a potential problem.^{40, 69} Several delivery mechanisms, such as the use of carbon monoxide releasing molecules (CORMs), are currently being developed to deliver CO to biological systems in a controlled manner.⁷⁰ However, use of CORMS can also result in dangerously elevated COHb levels, and their use is also complicated by their short half-lives, making their use as titratable therapeutic agents difficult. Similar to CO, H₂S is also naturally produced by the body as a signal gas and has similar pleiotropic biological effects which can be beneficial^{71, 72} even though at higher doses it is toxic.

In summary, exogenous CO, H₂S, and NO, either through inhalation or via use of hemoglobin-based oxygen carriers (HBOCs) or CO-releasing molecules (CORMs) or NO or H₂S releasing molecules have also been shown to have therapeutic value, including anti-inflammation, vasodilation or tissue protection. Key to their effectiveness is enhancing their bioavailability in plasma so that they are free to interact with the vasculature and with the organ and immune cells of the body to exert their beneficial effects. Thus, means to reduce their binding to either native hemoglobin or the hemoglobin of HBOCS are needed to optimize their bioavailability and to reduce their cytotoxic effects. While the use of acellular non-HBOC gas carriers such as intravenous perfluorocarbons (PFCs) can increase the solubility of and concentration of exogenously administered CO, NO, H₂S and O₂ in plasma, this enhanced concentration will not reduce the binding of these gases to hemoglobins, native or otherwise. Thus, in the absence of a solution to the sequestration of CO, H₂S and NO by native Hb and HBOCs, and the untoward side effects caused by this sequestration, the increased concentrations caused by PFCs are not helpful.

In addition, it is very likely that the avid binding of CO, H₂S, and NO to hemoglobin has reduced the ability to use these species as diagnostic measures in plasma or in exhaled air. Therefore, improving their bioavailability in plasma may render breath and plasma based measurements more accurate.

There is an urgent need for the development of methods for delivering medicinal gases such as CO, H₂S, and NO and for the successful use of blood substitutes without the attendant deleterious side effects which heretofore have accompanied their administration.

### SUMMARY OF THE INVENTION

The invention provides therapeutic and diagnostic methods i) to enhance the bioavailability of endogenously produced or exogenously provided therapeutic, medicinal gases (e.g. NO, N₂O, CO, H₂S, SO, SO₂ and O₂, or combinations thereof); and ii) to improve the efficacy of HBOCs with respect to delivery of oxygen to cells and tissues and to mitigate side effects.

With respect to i), an exemplary method involves the co-administration, with the medicinal gas, of at least one agent that changes the binding of the medicinal gas to Hb, usually by decreasing the binding and permitting the release of more gas than would be released in the absence of the agent. In other words, the agent causes an increase the rate and or quantity of release of gases from Hb, and hence an increase in the level of gases in circulation and their bioavailability. The gases are then available to exert their beneficial effect, e.g. smooth muscle relaxation, intracellular signaling, anti-inflammatory action etc., in the subject. In some embodiments, the agent is an allosteric modulator or effector that increases the P₅₀ of Hb. In some embodiments, administration of the agent is combined with administration of one or more PFCs and/or one or more HBOCs.

With respect to ii), an exemplary method involves the co-administration of at least one HBOC together with an agent that changes the binding affinity of the HBOC for O₂. Generally, as is the case for Hb as described above, the affinity is decreased, permitting the release of more O₂ than would be released in the absence of the agent. This results in an increase in the level of O₂ in circulation and its bioavailability e.g. for tissue oxygenation, in the subject. In some embodiments, the agent is an allosteric modulator or effector that increases the P₅₀ of Hb. Without being bound by theory, it appears that such allosteric modifiers also surprisingly exert similar effects on HBOCs. In other words, the allosteric agents appear to also cause an increase in P₅₀ of the HBOC, resulting in more O₂ being released from the HBOC and delivered to cells and tissues of a subject, thereby preventing or overcoming the limitations and untoward side effects of HBOC administration according to hitherto known methods. The agent appears to cause a shift in the equilibrium distribution of bound vs free gas, in favor of free, bioavailable gas. In addition, co-administration of an HBOC and such an agent also surprisingly increases the ability of the HBOC to release other medicinal gases (e.g. NO, N₂O, CO, H₂S, SO, SO₂, etc.), and in some embodiments of the method, medicinal gases are co-administered with the HBOC. This latter point is particularly important in regards to NO where its binding with HBOCs have resulted in complications. The same holds true for mixtures of e.g. erythrocyte Hb and HBOCs.

The administration of Hb/HBOC modulating agents can be used for both therapeutic and diagnostic purposes.

Embodiments of the invention provide methods of using agents such as modifiers of Hb and/or HBOCs (e.g. allosteric modifiers which increase the P₅₀ of the Hb or HBOC) in at least the following exemplary applications:

1) Enhance the bioavailability of endogenously produced CO, NO, H₂S, O₂, and other medicinal gases either alone or in combination, by reducing their binding to native hemoglobin for therapeutic and/or diagnostic purposes.

2) Enhance the bioavailability of exogenously produced or exogenously administered CO, NO, H₂S, O₂, and other medicinal gases either alone or in combination, for therapeutic and/or diagnostic purposes.

3) Enhance the bioavailability of exogenously stored or produced CO, NO, H₂S O₂, and other medicinal gases either alone or in combination, or for diagnostic purposes when they are used as a part of an HBOC delivery strategy for diagnostics or therapeutic purposes.

4) Enhance the therapeutic efficacy of HBOCs by reducing their propensity to bind NO H₂S, CO, and other medicinal gases.

5) Enhance the therapeutic or diagnostic efficacy of HBOCs by increasing their P₅₀ to enhance the off-loading of oxygen.

6) Enhancing the therapeutic or diagnostic efficacy of CO, NO, H₂S, O₂ and other medicinal gases in therapeutics and diagnostics e.g. in combination with perfluorocarbon administration with and without the use of HBOCs.

### BRIEF DESCRIPTION OF THE DRAWINGS

FIG. 1. Structure of RSR-13.

FIG. 2A-C. The effect of various concentration of RSR-13 on the OECs of Hb. (A) Human blood (hct 30%). (B) Swine blood (hct 30%). Round dot (), line (-), triangle (▴) and square dot (▪) are 0, 1, 2 and 5 mM RSR-13. (C) HBOC-1 (13.4 g/dL). Square dot (▪), round dot (), and triangle (▴) are 0, 2 and 5 mM RSR-13, respectively.

FIG. 3A-C: Effect of RSR-13 on the spectroscopic properties of cell free (A) human Hb; (B) swine Hb; and (C) HBOC-1 as monitored by UV-Viz spectroscopy in Soret region (417 nm). Round dot (); Square dot (▪); solid line (-) and long dashed line (- - - )are 0, 1 mM, 2 mM and 5 mM RSR-13, respectively.

### DETAILED DESCRIPTION

The invention provides methods to enhance the bioavailability of endogenously produced or exogenously provided medicinal gases (e.g. NO, CO, H₂S and O₂), and to improve the efficacy of HBOCs with respect to delivery of oxygen to cells and tissues and to decrease harmful NO binding to HBOCs. Exemplary methods involve the co-administration, with the medicinal gas and/or the HBOC, and/or a PFC, of an agent that changes the binding of a medicinal gas for Hb and/or the HBOC. Generally, the agent decreases the binding affinity of the gas for Hb and/or the HBOC, making it easier for the gas to be released into circulation, or, conversely, making it more difficult for the Hb or HBOC to sequester the gas. In some embodiments, the agent increases the P₅₀ of Hb and/or the HBOC for oxygen. In some embodiments, the agent is an allosteric modulator of Hb and/or HBOCs. In the case of the provision of medicinal gases or endogenously produced gases, concerted administration of such an agent causes Hb to bind the gases with lower affinity, thus increasing the rate and amount of release of gases from the Hb, and increasing the level of bioavailable gases in circulation. The gases are then available to exert their beneficial effect, e.g. smooth muscle relaxation, intracellular protection, anti-inflammation, etc., in the subject. With respect to HBOCs, the change in gas binding affinity of the HBOC (e.g. a P₅₀ increase for O₂) caused by the concerted administration of such an agent results in more O₂ (or other gas, e.g. if CO, NO, H₂S, SO₂, etc. are also administered) being released from the HBOC and available for delivery to cells and tissues of the subject, thereby preventing or overcoming the limitations and untoward side effects of HBOC administration which result when prior art administration methods are used. It also would decrease the harmful binding of NO to the HBOC which as been associated with harmful side-effects.

By “changing” the binding of a gas to a gas carrier such as Hb we mean that the binding affinity of the gas for the carrier in the presence of the agent differs from the binding affinity of the gas for the carrier in the absence of the agent. Generally, the change is a change of at least about 5, 10, 20, 30, 40, 50 60, 70, 80, 90, or 100%, and may be a change of about 1, 2, 3, 4, 5, 6, 7, 8, 9, 10, 20, 30, 40, 50, 60, 70, 80, 90 or 100-fold, or even more (e.g. a 500- or even 1000-fold change. Advantageously, the change is a decrease in binding affinity, which results in a greater release of the gas from the carrier into the surrounding milieu or a decrease in the uptake of the gas (e.g. a deleterious or poisonous gas) from the surrounding milieu to Hb as a gas carrier.

“P₅₀” refers to the partial pressure of a gas such as oxygen at which a gas carrier (e.g. an oxygen carrier such as Hb or an HBOC, or a mixture of these) is 50% saturated with the gas. Thus, lower values indicate greater affinity for the gas, i.e. a decreased tendency to release the gas, or, conversely, an increased tendency to retain bound gas. On the other hand, an increase in P₅₀, as may be caused by the agents employed as described herein, results in an increased tendency of the carrier to release the gas, or conversely, a decreased tendency to retain bound gas e.g. oxygen.

By “exogenous” administration, we mean a type of administration which includes but is not limited to, for example, intravenous, dermal and oral administration, inhalation, etc.

By “endogenous” administration, we mean that the gas is technically produced by the body, for example, in response to inadvertent trauma. Alternatively, the “endogenous” production of the gas may be purposefully induced, e.g. by the deliberate application of an exogenous stimulus or means such as mechanically through a cuff or tourniquet which causes ischemia and/or reperfusion, or by mechanical means such vibrating, or by providing precursors to the gas intravenously, etc.

The “bioavailability” of a therapeutic gas as used herein refers to the degree to which or rate at which a therapeutic agent such as a medicinal gas is absorbed and/or becomes available at the site of physiological activity to exert its effect.

By co-administration or administered together, we mean that two (or more) agents are administered so as to both (or all) be present in a subject at the same time, or at least at overlapping times, or at least so that the effect of each agent is still present in the subject when the other(s) are administered. The agents may literally be administered at the same time (either in a single composition, or in separate compositions), or sequentially within a relatively short period of time, or one may be administered more or less continuously and the other(s) administered during the time of administration, or one or more of the agents may be in a sustained, long acting formulation, etc.

Medicinal or therapeutic gases that may be employed in or administered according to the methods of the invention include but are not limited to: NO, CO, hydrogen sulfide (H₂S), nitrogen dioxide (N₂O), sulfur monoxide (SO) and sulfur dioxide (SO₂), and combinations of these. NO and several sulfur containing gases are known to have beneficial and/or protective effects.⁷¹⁻⁷⁶ For example, H₂S is known to be endogenously produced and to have profound protective effects on cells when administered, e.g. via inhalation or injection.

An administrable source of a medicinal gas may be, for example: medical grade NO and CO are available for direct inhalation and can be mixed with oxygen; a PFC that is loaded with one or more gases of interest may be administered; CO-releasing molecules (CORMs) such as [Mo(CO)₃(histidinato)]Na are water soluble and when injected release CO; compounds that break down and release e.g. H₂S or another medicinal gas in an aqueous environment such as in blood or plasma in vivo, etc. may be used; injection of NaHS intravenously produces H₂S as is described in a review by Czabo;⁷⁷ injection of NO donors such as nitrates and Naproxen-NO will subsequently increase circulating levels of NO as described in a review by Thatcher and colleagues.⁷⁸

Compounds which may be used in the practice of the invention include but are not limited to:

1. Allosteric effectors of hemoglobin such as compounds with general Formula I, and functional variants, analogues, isomers and salts thereof:

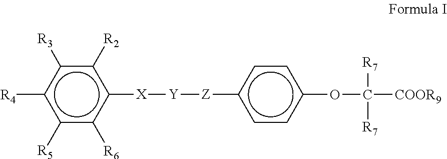

wherein X, Y and Z may each be CH₂, NH, or O, R₂₋₆ are either hydrogen, halogen, or a substituted or unsubstituted C₁, C₂, or C₃ alkyl group and these moieties maybe the same or different, or alkyl moieties of aliphatic or aromatic rings incorporating two of the R₂₋₆ sites, R₇₋₈ are hydrogen, methyl, or ethyl groups and these moieties may be the same or different, or alkyl moieties as part of an aliphatic ring connecting R₇ and R₈, and R₉ is a hydrogen, lower alkyl such as methyl, ethyl or propyl, or a salt cation such as sodium, potassium, or ammonium.

Compounds of this type and variants and analogues thereof are described in U.S. Pat. No. 5,122,539; 5,248,785; 5,250,701; 5,290,803; 5,525,630; 5,591,892; 5,648,375; 5,661,182; 5,677,330; 5,705,521; 5,731,454; 5,827,888; 5,872,282; and 5,927,283, the complete contents of each of which are hereby incorporated by reference. An exemplary compound of this type is “RSR-13”. RSR-13 is also known by its International Nonproprietary Name (INN) “efaproxiral”; its IUPAC designation is 2-[4-[2-[(3,5-dimethylphenyl)amino]-2-oxoethyl]phenoxy]-2-methylpropanoic acid); and its formula is presented in Formula 2:

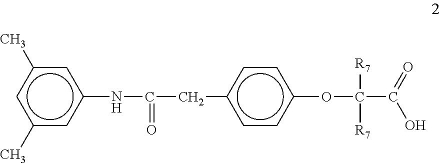

2. Myoinostitol trispyrophosphate (CAS Number:802590-64-3; formula: C₆H₁₂O₂₁P, also known as “inositol tripyrophosphate”, “ITPP”, “myo-inositol”, “1,6:2,3:4,5 tripyrophosphate”, etc.); is depicted in Formula 3, and functional variants, analogues, isomers and salts thereof.

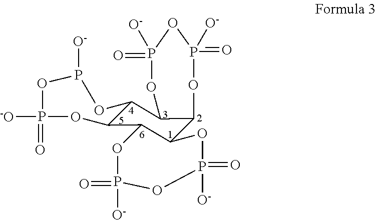

Various forms and uses of ITPP are described, for example, in U.S. Pat. No. 7,919,481; 7,745,423; 7,648,970; and 7,618,954; and US patent applications 2010/0267674; 20100029594; 2010/0029593; 2009/0029951; 2008/0312138; 2007/0135389; 2006/0258626; 2006/0241086; and 2006/0106000, the complete contents of each of which are hereby incorporated by reference.

In addition, newer allosteric modifiers such as those produced by Normoxys (see the web site located at www.normoxys.com) are also envisioned to be effective.

HBOCs that can be used in the practice of the invention include but are not limited to: HBOC-1, and functional variants thereof, as well as those which are described in the following: U.S. Pat. No. 4,001,401 to Bonson et al., and U.S. Pat. No. 4,061,736 to Morris et al., the complete contents of each of which are herein incorporated by reference, describe different approaches to producing viable blood substitutes which may be used in the practice of the present invention. U.S. Pat. Nos. 7,803,912; 7,642,233; 7,307,150; 7,005,414; 6,894,150; 6,812,328; 6,808,898; 6,803,212; 6,506,725; 6,486,123; 6,172,039; 6,083,909; 6,072,072; 6,005,078; 5,962,651; 5,955,581; 5,674,528; and 5,661,124, among others, describe various HBOCs and/or methods of making HBOCs that are suitable for use in the present invention, as do US patent applications 20100323029; 20080305178; 20070172924; 20050113289; 20040029780; 20030181358; among others, and the complete contents of each of these patents and patent application are herein incorporated by reference in entirety. Any HBOC, the P₅₀ of which can be increased by a modifying agent such as an allosteric modifying agent, may be used in the practice of the invention. The complete contents of each of the foregoing patents and patent applications are hereby incorporated by reference in entirety. While there are no currently approved HBOC's for use in humans or animals, these are expected to be forthcoming and in fact, with the use of the described invention may be forthcoming sooner than without the described invention.

The active agents that are administered according to the invention (i.e. the agents which increase the P₅₀ of Hb and/or an HBOC) and the HBOCs themselves are administered as compositions which are suitable to their properties (i.e. to maintain functionality) and to the desired mode of administration and action. The compositions generally include a pharmacologically suitable carrier. The preparation of such compositions is well known to those of skill in the art. Typically, such compositions are prepared either as liquid solutions or suspensions, or as solid forms such as tablets, pills, powders and the like. Solid forms suitable for solution in, or suspension in, liquids prior to administration may also be prepared, and preparations may also be emulsified. The active ingredients may be mixed with excipients which are pharmaceutically acceptable and compatible with the active ingredients. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. If it is desired to administer an oral form of the composition, various thickeners, flavorings, diluents, emulsifiers, dispersing aids or binders and the like may be added. The composition of the present invention may contain any such additional ingredients so as to provide the composition in a form suitable for administration. The final amount of active agent in the formulations may vary. However, in general, the amount will be from about 1-99%.

The compositions (preparations) may be administered by any of many suitable means which are well known to those of skill in the art, including but not limited to by injection, inhalation, orally, intravaginally, intranasally, by ingestion of a food or probiotic product, topically, as eye drops, via sprays, etc. In preferred embodiments, the mode of administration is by injection. In addition, the compositions may be administered in conjunction with other treatment modalities such as antibiotic agents, and the like.

The amount of Hb/HBOC modulating agent that is administered is typically in the range of from about 1 mg/kg to about 500 mg/kg, and is preferably from about 25 mg/kg to about 300 mg/kg, or about 50 mg/kg to about 120 mg/kg, or 75 mg/kg to about 100 mg/kg. The amount of HBOC that is typically administered in the practice of the invention is in the range of from about 100 cc to about 2000 cc, and is preferably from about 250 cc to about 500 cc. If PFCs are concurrently administered, the amount is generally in the range of from about 0.25 cc/kg to about 10 cc/kg, and is preferably from about 2 cc/kg to about 5 cc/kg. These doses represent bolus doses but may be modified and increased to be provided as continuous infusions while maintaining continuous therapeutic concentrations of the above agents to achieve the desired effects. The doses above of course may changes as new studies are performed to optimize combinations for the various diagnostic and therapeutic purposes as they relate to specific diseases. It is also recognized that newer formulations may arise which change the volumes of agents provided.

The methods of the invention can be used to treat any patient or subject suffering from or likely to suffer from a disease or condition which can be prevented, treated, cured, or ameliorated (i.e. disease symptoms are abated) by increasing the concentration and/or the bioavailability of a beneficial gas in the circulatory system of the subject. Such patients or subjects are generally mammals, frequently humans, although this need not always be the case. Veterinary applications of this technology are also encompassed, e.g. to treat companion pets (dogs, cats, etc.), domestic animals such as horses, cattle, goats, sheep, pigs, etc., wildlife in captivity or in preserves (especially rare or endangered species, or those used for breeding purposes), and others that may benefit from the practice of the invention.

Various embodiments or scenarios of use of the methods of the invention include but are not limited to:

1) Patients who have incurred an acute or chronic illness or injury in which the body is producing additional endogenous NO and/or H₂S, and/or CO and/or other therapeutic gas (e.g. in order to maintain microvascular blood flow and/or combat inflammation and other cell damaging activities) would be given either a bolus or intermittent, or continuous infusion of an agent such as RSR-13 over suitable time periods. The agent acts to enhance the bioavailability of the endogenously produced NO and CO by reducing their binding to native hemoglobin. At the same time, tissue oxygenation is concurrently enhanced either with or without the presence of supplemental oxygen. Examples of such chronic or acute illnesses or injuries include but are not limited to hemorrhagic and traumatic shock, severe infection (bacterial and otherwise), severe sepsis, and septic shock, cardiac arrest and cardiogenic shock, severe burns and wounds, complex surgeries such as transplant surgeries, Crohn's disease, radiation poisoning, traumatic brain injury, stroke, myocardial infarction, vasoocclusive crisis, severe respiratory distress from asthma, chronic obstructive pulmonary diseases, acute respiratory distress syndrome, pulmonary hypertension, preeclampsia, eclampsia, malaria, influenza, organ transplant, coronary heart disease, cerebrovascular disease, hypertension, arthritis, cancer, and others.

2) Patients who have incurred and acute illness or injury or who have a chronic condition that would benefit from the administration of exogenous NO, H₂S and/or CO or other therapeutic gas with or without supplemental oxygen would have an intravenous bolus or intermittent or continuous infusion of an agent such as RSR-13 in conjunction with receipt of the exogenous gases. This embodiment may be further modulated by the concurrent administration of perfluorocarbon emulsions allowing for more NO, CO, H₂S, O₂ and combinations thereof to be carried by plasma, while still reducing their binding to hemoglobin. In fact, NO, CO, H₂S, and/or O₂ may be premixed with the PFC to allow their administration intravenously without the need for inhalation of these gases if desired. Examples of such acute illnesses or injuries or chronic conditions include but are not limited to: hemorrhagic and traumatic shock, severe infection (bacterial and otherwise), severe sepsis, and septic shock, cardiac arrest and cardiogenic shock, severe burns and wounds, complex surgeries such as transplant surgeries, Crohn's disease, radiation poisoning, traumatic brain injury, stroke, myocardial infarction, vasoocclusive crisis, severe respiratory distress from asthma, chronic obstructive pulmonary diseases, acute respiratory distress syndrome, pulmonary hypertension, preeclampsia, eclampsia, organ transplant, malaria, influenza, coronary heart disease, cerebrovascular disease, hypertension, arthritis, cancer, and others.

3) Patients who require supplemental tissue oxygenation with an HBOC would be given an intravenous bolus or intermittent or continuous infusion of an agent such as RSR-13 to increase the P₅₀ of both the HBOC and native hemoglobin with or without supplemental oxygen, thereby reducing CO, H₂S, and NO binding by the HBOC and native hemoglobin. Examples of illnesses or conditions which can benefit from supplemental tissue oxygenation with an HBOC include but are not limited to: hemorrhagic and traumatic shock, severe infection (bacterial and otherwise), severe sepsis, and septic shock, cardiac arrest and cardiogenic shock, severe burns and wounds, complex surgeries such as transplant surgeries, Crohn's disease, radiation poisoning, traumatic brain injury, stroke, myocardial infarction, vasoocclusive crisis, severe respiratory distress from asthma, chronic obstructive pulmonary diseases, acute respiratory distress syndrome, pulmonary hypertension, preeclampsia, eclampsia, organ transplant, malaria, influenza, coronary heart disease, cerebrovascular disease, hypertension, arthritis, cancer, and others.

The methods of the invention may also be used as or in conjunction with diagnostic methods. For example, it is well-known that the production of CO, H₂S, and NO is increased in subject with certain types of illnesses or injuries e.g. sepsis, hemorrhage, trauma, infections, asthma, vasooclusive crisis, etc. Thus, the detection and measurement of CO, H₂S, and NO can serve as an indicator of or as confirmation of the presence of injury or disease. However, such measurements are hampered by sequestration of CO, H₂S, and NO by Hb (and by HBOCs, when administered). The administration of Hb modulating agents as described herein decreases sequestration of CO, H₂S, and NO and thus increases their concentrations in media such as plasma (the acellular component of blood), air (e.g. breath), etc. and renders the level of gases more detectable. The detection of high levels of these or other Hb binding gases may indicate the presence of suspected or otherwise asymptomatic injury or disease, or may confirm the presence of injury or disease. Such methods may also be used to monitor the progress or stage of injury or disease, or to monitor the efficacy of disease/injury treatment and healing, or lack thereof. For example, the amount of NO, H₂S, and/or CO in exhaled breath or in blood or plasma samples from a patient who is believed to be acutely ill or injured can be measured after administration of an agent such as RSR-13. The measured levels are compared to predetermined control values measured under the same conditions (e.g. from healthy individuals who do not have the injury or disease). If the values measured in the patient exceed those of the control value, then a diagnostician may conclude that the patient has the injury or disease that is characterized by or which elicits the production of NO, H₂S, and/or CO. However, if the measured values are not higher than the control values (e.g. if they are the same as the control values, within experimental error), then one would likely conclude that the patient does not have the disease or injury in question. In addition, if the values are lower than the control values, one might conclude that the patient suffers from a disease or condition that impairs the body's ability to produce CO, H₂S, and NO. This may be an indication of a situation in which the patient should receive exogenous CO, H₂S, and/or NO therapy. Such strategies may even be more diagnostic when coupled with additional provocative testing. For example, new cardiovascular testing is now being promoted examining the vascular response to vasocclusive testing. In such test, a blood pressure cuff is inflated in the upper portion of an extremity for a period of time to reduce blood flow to tissues below the level of cuff inflation. Upon deflation, the vascular response is examined by such technologies as photoplethysmography, temperature, or near-infrared spectroscopy. Such testing is demonstrating the ability to help understand who may be at risk for cardiovascular disease. However, none of these test take into account the dynamics of hemoglobin binding to NO or CO. Thus, the addition of RSR-13 or similar allosteric effectors to these or other tests that transiently produce changes in NO, H₂S, or CO dynamics may add diagnostic value. This strategy may be applied to other biologic gases that bind to hemoglobin as well.

The invention also provides methods of treating poisoning by gases such as CO, H₂S and NO₂. The method involves co-administering to subject or patient in need thereof (i.e. one suffering from gas poisoning) i) an agent that changes the binding of the gas to Hb (e.g. an allosteric modulator of Hb such as RSR13); and ii) one or both of an HBOC and a PFC. As a result of administering the agent, native hemoglobin and, if present, the HBOC, release more O₂ into circulation, while the agent also prevents the poisoning gas from binding to Hb and/or the HBOC. As a result, more oxygen is delivered to tissues and more poisonous gas is flushed from the system.

The following examples serve to illustrate various embodiments of the invention but should not be interpreted so as to limit the scope of the invention in any way.

### EXAMPLES

### Example 1

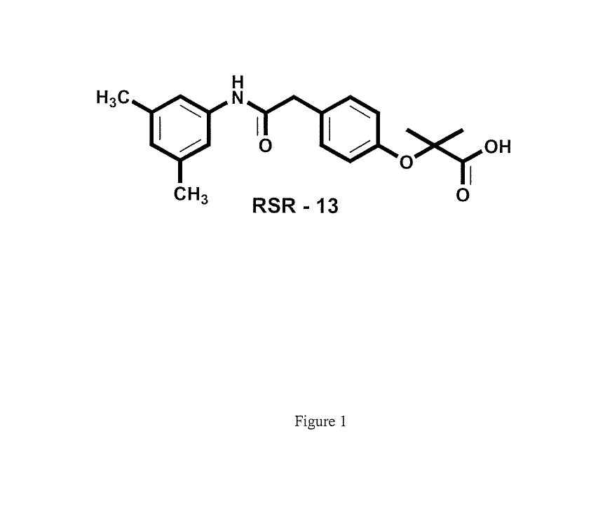

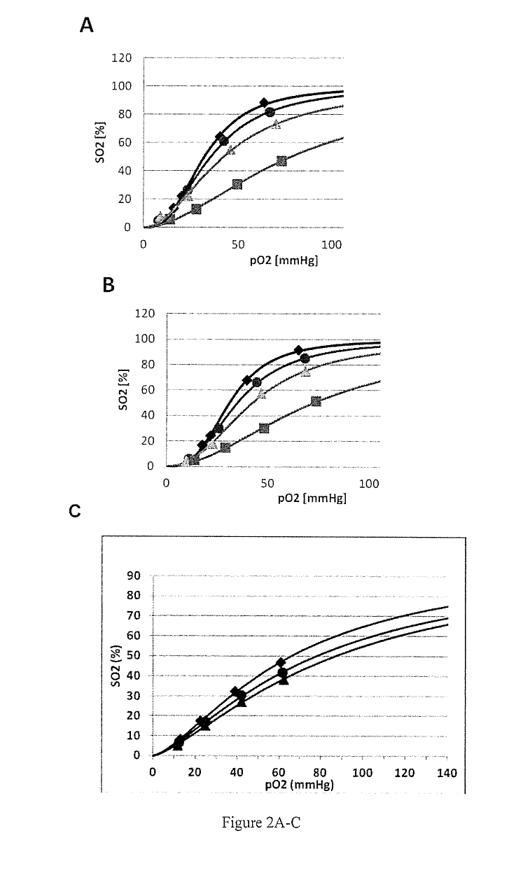

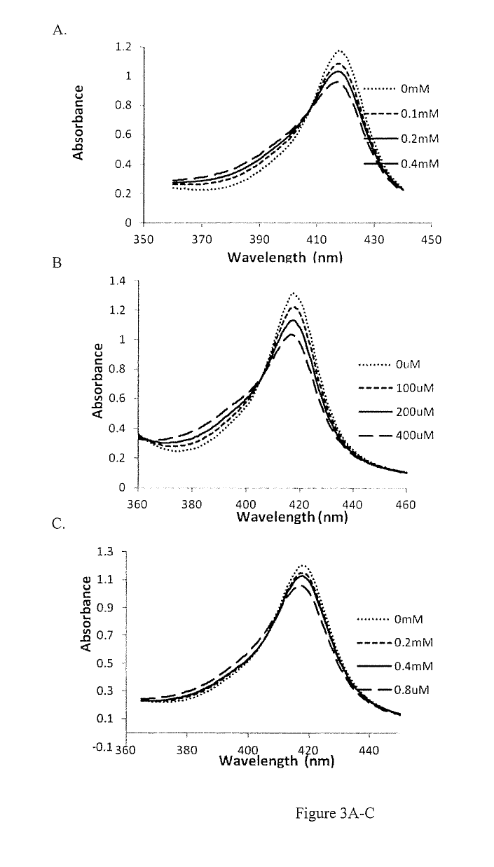

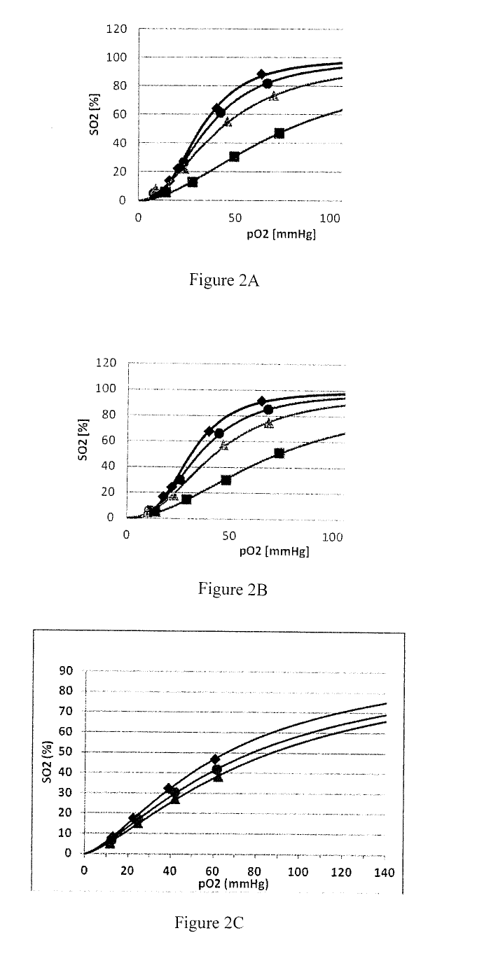

We have determined the effect of RSR-13 (FIG. 1) on the spectroscopic properties of the NO derivatives of cell free human Hb (hHb) and swine Hb (swHb), as well as the NO derivative of HBOC-1. HBOC-1 (Oxyglobin® obtained from BioPure Inc) is a highly purified bovine Hb that is crosslinked with gluteraldehyde. We have also measured the oxygen saturation levels of swine whole blood or erythrocyte (erythswHb), human whole blood or erythrocyte (erythhHb), HBOC-1, and solution mixtures of HBOC-1 and erythswHb in the presence of RSR-13. Finally, we have measured the COHb levels of erythswHb and HBOC-1 in the presence of RSR-13. We observed significant effect of RSR-13 on Hb oxygen, NO and CO binding properties.

### Experimental Section

### Materials

Human hemoglobin (hHb) or swine Hb (swHb) was purified from discarded blood samples following published procedure⁷⁹ and dialyzed in 0.1 M HEPES buffer containing 0.1 M NaCl, pH 7.0. The use of the human blood sample is in accordance with regulations of the IRB for Protection of Human Subjects. GMP grade RSR-13 was obtained at Virginia Commonwealth Univ. The compounds were solubilized with 0.1 mM HEPES buffer containing 0.1 mM NaCl, pH7.0 for absorbance studies. HBOC-1 (Oxyglobin® from BioPure Inc) was studied. Oxyglobin® was approved for veterinary use but is no longer available.

### Effect of RSR-13 on the Oxygen Affinity of ErythswHb, ErythhHb and HBOC-1

The effect of RSR-13 on the oxygen affinity of erythswHb and erythhHb, and HBOC-1 was determined using multiple-point tonometer. RSR-13 (1, 2 or 5 mM) was incubated with blood from swine or human (hct ˜30%) or HBOC-1 (13.4 g/dL) for 30 minutes. The Hb-compound mixture was then further incubated in IL 237 tonometers (Instrumentation Laboratories, Inc. Lexington, Mass.) for 5 minutes at 37° C. to equilibrate at oxygen tensions of 6 mmHg or 20 mmHg or 40 mmHg or 60 mmHg. Following, the sample is aspirated into an IL 1420 Automated Blood Gas Analyzer and an IL 682 Co-oximeter (Instrumentation Laboratories) or ABL 700 Blood Gas Analyzer (Radiometer, Westlake, Ohio) to determine the pH, pCO₂, pO₂, and Hb oxygen saturation values (SO₂). The measured values of pO₂ and SO₂ were then subjected to a non-linear regression analysis using the program Scientist to estimate the P₅₀ and Hill coefficient values (cooperativity of oxygen binding; n₅₀).

### Effect of RSR-13 on the Oxygen Affinity of Solution Mixtures of ErythswHb and HBOC-1.

RSR-13 (2 mM) was incubated with erythswHb (hct of 15 or 20%)/HBOC-1 (13.4 g/dL) solution mixture in a volume ratio of 75:25 and 50:50 for 30 minutes. The Hb-compound mixture was then analyzed for their P₅₀ and Hill coefficient values as described above.

Effect of RSR-13 on the Spectroscopic Properties of NO Derivatives of HBOC-1 and Cell Free Hb from Human and Swine

Effect of RSR-13 on the absorption spectra in the soret region of the nitrosylated derivatives of cell free Hb from human and swine, and HBOC-1 were recorded at 37° C. using HP Agilant 8453 Spectrophotometer using 1 cm path length cuvette. Solid Na dithionite (2 mg/mL) was added to the Hb to make the fully deoxygenated Hb, followed by addition of solid diethylamine NONOate (1.3 mg/mL) to make the NOhHb or NOswHb, or NOHBOC-1 derivatives. RSR-13 (20 uM-800 uM) were added to 65 ug of the NO derivatives and the spectrum monitored in the soret region at 417 nm.

### Results

### RSR-13 Reduces Hb and HBOC-1 Affinity for Oxygen

RSR-13 was tested for its ability to decrease the oxygen affinity of human and swine blood and HBOC-1, and quantified by its ability to increase P₅₀ (partial pressure of oxygen at 50% Hb saturation). Allosteric effectors that decrease Hb oxygen affinity increase the P₅₀ (right-shift the OEC) relative to the control. Values of P₅₀ and n₅₀ for oxygen binding to the Hbs are shown in Tables 1-3. In the absence of RSR-13, the P₅₀ of swine and human blood is ˜31 mmHg and n₅₀ of ˜3.0. The P₅₀ and n₅₀ of HBOC-1 are 58.5 mmHg and 1.2. Addition of RSR-13 affects the oxygen binding properties of all Hbs, causing dose-related increase in P₅₀ and/or decrease in n₅₀ (Table 1-3, FIG. 1 ). At 2 mM RSR-13 concentration, the ΔP₅₀ shift is significantly more in HBOC-1 compared to the whole blood from swine or human (15.1 vs. ˜10.5 mmHg) mmHg), but at the higher RSR-13 concentration of 5 mM, the P₅₀ shift in the blood is almost twice as observed in the HBOC-1 (˜43 vs. 25 mmHg). As expected, the Hb from swine and human exhibits significant cooperativity (˜3) compared to almost no cooperativity in HBOC-1 (˜1.2) (FIG. 1A-C). RSR-13 has a major effect on the cooperativity of the swine and human Hb as the shape of the OEC changed from sigmoid to hyperbolic with increased RSR-13 concentration (FIG. 1A,B), while HBOC-1 barely exhibited any change in the Hill coefficient (FIG. 1C). Table 3 also shows the P₅₀, ΔP₅₀ and n₅₀ of swine blood at hct of 30%, 20% and 15%, respectively with 2 mM RSR-13. There is an inverse relationship between hematocrit level and P₅₀ shift. There were no significant differences between the n₅₀ values.

TABLE 1
Effect of various concentrations of RSR-13 on human and swine
whole blood oxygen affinity^{a}

| | Human blood (hct 30%) | | | Swine blood (hct 30%) | | |
|---|---|---|---|---|---|---|
| | P₅₀ | ΔP₅₀ | | P₅₀ | ΔP₅₀ | |
| Conc | (mmHg) | (mmHg) | n₅₀ | (mmHg) | (mmHg) | n₅₀ |
| 0 | 31.8 ± 1.3 | — | 2.7 ± 0.3 | 30.9 ± 0.3 | — | 3.0 ± 0.1 |
| 1 mM | 35.1 ± 2.2 | 3.3 | 2.4 ± 0.2 | 34.5 ± 0.2 | 3.9 | 2.6 ± 0.3 |
| 2 mM | 42.0 ± 1.7 | 10.2 | 2.0 ± 0.3 | 41.5 ± 0.5 | 11.3 | 2.3 ± 0.0 |
| 5 mM | 78.0 ± 1.2 | 46.2 | 1.8 ± 0.1 | 72.1 ± 1.3 | 41.5 | 1.9 ± 0.1 |

^{a}Mean triplicate measurements.
^{b}P₅₀ is the O₂ pressure at which Hb is 50% saturated with oxygen.
^{c}ΔP₅₀ is P₅₀ of RSR-13 treated blood − P₅₀ of control.
n₅₀ is the cooperativity of oxygen binding at 50% Hb saturation with oxygen.
RSR-13 solubilized with water.

TABLE 2
Effect of various concentrations of RSR-13 on HBOC oxygen affinity^{a}

| | HBOC-1 (13.4 g/dL) | | |
|---|---|---|---|
| | P₅₀ | ΔP₅₀ | |
| Conc | (mmHg) | (mmHg) | n₅₀ |
| 0 | 58.5 ± 6.9 | — | 1.2 ± 0.3 |
| 2 mM | 73.6 ± 3.0 | 15.1 | 1.1 ± 0.3 |
| 5 mM | 83.5 ± 3.4 | 25.0 | 1.1 ± 0.3 |

^{a}Mean triplicate measurements.

TABLE 3
Effect of RSR-13 on swine blood oxygen affinity at different hematocrits^{a}

| | hct 30% | | hct 20% | | hct 15% | |
|---|---|---|---|---|---|---|
| RSR-13 | 0 | 2 mM | 0 | 2 mM | 0 | 2 mM |
| P₅₀ | 30.9 ± 0.3 | 41.5 ± 0.5 | 34.5 ± 1.1 | 51.3 ± 3.2 | 37.3 ± 1.2 | 62.3 ± 0.5 |
| n₅₀ | 3.0 ± 0.1 | 2.3 ± 0.0 | 3.0 ± 0.1 | 2.2 ± 0.1 | 3.1 ± 0.1 | 2.2 ± 0.2 |
| ΔP₅₀ | — | 11.3 | — | 16.8 | 25.0 | |

^{a}Mean triplicate or duplicate measurements

### RSR-13 Affects the Oxygen Affinity of Whole Blood and HBOC-1 Solution Mixtures

Shown in Tables 4 and 5 are the P₅₀ and n₅₀ of the erythswHb/HBOC-1 solution mixtures in the presence or absence of 2 mM RSR-13. Measurements were made for a 100:0, 75:25, 50:50 or 0:100% volume ratio of swine blood/HBOC-1 mixtures which was composed of either 15% or 20% hct whole blood in 13.4 g/dL of HBOC-1. In the absence of RSR-13, the data shows that the oxygen affinity of the various mixtures increased with increasing concentration of HBOC-1, while exhibiting decreasing cooperativity effect. Thus, although the mixed blood still retained cooperativity, it decreases with increasing concentration of the HBOC. In all experiments, including mixed and unmixed Hb, RSR-13 significantly reduced the affinity of the Hb for oxygen. The Hb oxygen affinity reduction with 2 mM addition of RSR-13 was greater in the unmixed swine blood and the mixed samples when compared to the pure HBOC-1.

TABLE 4
Effect of RSR-13 on erythswHb/HBOC-1 mixture oxygen affinity at 20% hct^{a}

| | | | Blood/HBOC | | Blood/HBOC | | | |
|---|---|---|---|---|---|---|---|---|
| | Blood (100%) | | (75:25) | | (50:50) | | HBOC | |
| RSR-13 | 0 | 2 mM | 0 | 2 mM | 0 | 2 mM | 0 | 2 mM |
| P₅₀ | 34.5 ± 1.1 | 51.3 ± 3.2 | 43.4 ± 0.7 | 61.5 ± 3.6 | 47.6 ± 4.0 | 68.0 ± 4.2 | 58.5 ± 6.9 | 73.6 ± 3.0 |
| n₅₀ | 2.95 ± 0.1 | 2.2 ± 0.1 | 2.6 ± 0.2 | 2.1 ± 0.1 | 2.2 ± 0.1 | 1.8 ± 0.3 | 1.2 ± 0.3 | 1.1 ± 0.3 |
| ΔP₅₀ | 16.8 | | 18.1 | | 20.5 | | 15.1 | |

^{a}Mean duplicate or triplicate measurements.
Hct of swine blood used is 20%.
Concentration of HBOC-1 used is 13.4 g/dL

TABLE 5
Effect of RSR-13 on erythswHb/HBOC-1 mixture oxygen affinity at 15% hct^{a}

| | | | Blood/HBOC | | Blood/HBOC | | | |
|---|---|---|---|---|---|---|---|---|
| | Blood (100%) | | (75:25) | | (50:50) | | HBOC | |
| RSR-13 | 0 | 2 mM | 0 | 2 mM | 0 | 2 mM | 0 | 2 mM |
| P₅₀ | 37.3 ± 1.2 | 62.3 ± 0.5 | 47.6 ± 2.3 | 75.1 ± 5.6 | 52.8 ± 2.6 | 74.0 ± 2.1 | 58.5 ± 6.9 | 73.6 ± 3.0 |
| n₅₀3. | 1 ± 0.1 | 2.2 ± 0.2 | 2.3 ± 0.2 | 1.7 ± 0.1 | 1.9 ± 0.1 | 1.5 ± 0.1 | 1.2 ± 0.3 | 1.1 ± 0.3 |
| ΔP₅₀ | 25.0 | | 27.6 | | 21.2 | | 15.1 | |

^{a}Mean duplicate or triplicate measurements.
Hct of swine blood used is 15%.
Concentration of HBOC-1 used is 13.4 g/dL

### RSR-13 Reduces Hb and HBOC-1 Affinity for NO

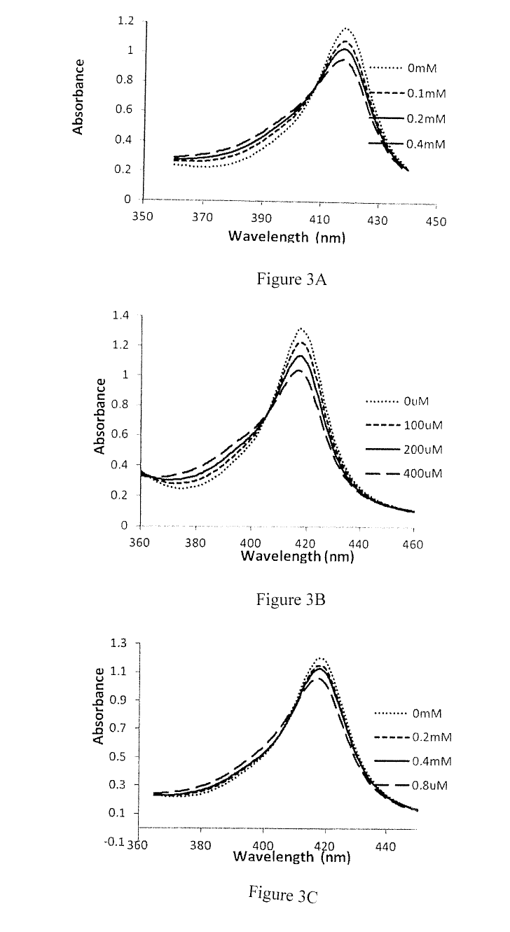

NOHb has a characteristic absorbance band at 419 nm in the Hb Soret region, which have been used to study the spectroscopic properties of NO derivative of Hb in the presence of IHP and BZF.⁸⁰ FIG. 3 show the spectra of NO derivatives with increasing concentration of RSR-13 (20 uM-800 uM). Table 6 is the % shift in the peak height, and shows RSR-13 significantly decreasing the intensity of the absorbance band in the soret region in a concentration dependent manner. The effect is similar for the Hb from swine and human, and more pronounced than observed with both HBOC-1. At 400 μM of RSR-13, there is about 20% decreased in peak intensity for the human and swine Hb, compared to about 10% for HCOC-1.

TABLE 6
Percentage (%) decrease in absorbance intensity at 417 nm of cell free
Hb and HBOC-1

| Conc | Human Hb | Swine Hb | HBOC-1 |
|---|---|---|---|
| 20 uM | 3.2 ± 0.4 | 3.3 ± 0.4 | — |
| 100 uM | 8.9 ± 0.3 | 7.9 ± 0.4 | 4.7 ± 0.2 |
| 200 uM | 13.2 ± 0.3 | 14.9 ± 0.3 | 6.1 ± 0.3 |
| 300 uM | 16.5 ± 0.4 | 17.1 ± 0.5 | 7.8 ± 0.5 |
| 400 uM | 19.4 ± 0.5 | 21.9 ± 0.3 | 9.7 ± 0.3 |
| 800 uM | — | — | 15.5 ± 0.5 |

The results are the means ± S.E. for 2 measurements. Concentration of the Hb is 65 ug.

### RSR-13 Reduces COHb Levels in Swine Blood

To determine whether RSR-13 will reduce COHb levels in swine blood or HBOC-1, we incubated HBOC-1 (13.4 g/dL) that has been exposed to CO to form ˜51% COHb, and swine whole blood (hct of 30%) that has been exposed to CO to form ˜52 or 10% COHb concentrations with 2 mM RSR-13. The COHb levels of the swine blood and HBOC-1 were tested after 30 minutes, and the results shown in Table 7. RSR-13 was able to reduce the amount of COHb by about 16% in the swine blood in the experiment starting with either 10% or 52% COHb level, and about 14% in the HBOC-1.

TABLE 7
Effect of RSR-13 on COHb levels of HBOC-1 (13.4 g/dL) and swine
blood (hct 30%)^{a}

| | | COHb levels | |
|---|---|---|---|
| Hb | control | 2 mM RSR-13 | % COHb decrease |
| Swine blood | 52.4 ± 1.5 | 44.2 ± 1.0 | 15.6% |
| Swine blood | 10.2 ± 0.1 | 8.5 ± 0.3 | 16.6% |
| HBOC-1 | 51.1 ± 0.6 | 44.1 ± 0.7 | 13.7% |

^{a}Mean duplicate measurements.

### DISCUSSION

### RSR-13 Increases the Oxygen Affinity of Both Whole Blood and HBOC

In the OEC study, RSR-13 induced significant reduction in oxygen affinity in all Hbs, including from blood and HBOC, although to varying degree. The results shows that the HBOC-1 is more efficient in releasing oxygen, that is have lower oxygen affinity, when compared to the pure erythrocyte, whether from human or swine. Addition of RSR-13 significantly increased the release of more oxygen from all the Hbs. Interestingly, at 2 mM concentration of RSR-13, HBOC-1 shows more efficiency in oxygen release than the erythrocyte Hb, however at 5 mM, the converse is true. It is also apparent that unlike the erythrocyte Hb, the HBOC-1 does not exhibit any significant cooperativity, and as expected, addition of the RSR-13 has limited effect on the Hill coefficient. Most likely the cross-linking of HBOC-1 introduces conformational constraint in the blood substitutes that could explain the absence of cooperativity, as well as their reduced responses to RSR-13. Thus the ability to effect release of oxygen and other gases from HBOC's is unexpected and not obvious prior to these experiments. Another notable observation is that at 2 mM there is a significant negative relationship between the hematocrit of blood and ΔP₅₀, most likely due to less Hb molecules. Similar effect is also observed between the estimated P₅₀ value of the control and the hematocrit, although not quite as significant.

### RSR-13 Modulates the Oxygen Affinity of Blood/HBOC-1 Mixtures

Since patients receiving HBOC would certainly have erythrocytes in the circulatory system, we decided to study the oxygen affinity behavior of blood/HBOC-1 mixtures in the presence of RSR-13 and compare it to the unmixed samples. In all experiments, including mixed and unmixed Hb, RSR-13 significantly reduced the affinity of the Hb for oxygen. The reduced Hb affinity was significantly greater in the erythrocyte and the mixed when compared to the pure HBOC. Additionally, unlike the unmixed HBOC-1, the mixed samples showed some cooperativity effect. These observations suggest that the behavior of the mixed samples is closer to the unmixed blood. The data also shows that in the presence of RSR-13 the pure erythrocyte at hct 20 or 15% and its mixtures with HBOC-1 are more efficient in oxygen release than pure HBOC-1. In contrast, and as shown earlier, HBOC-1 is more efficient in releasing oxygen with 2 mM RSR-13 when compared to whole blood at 30% hct. The data suggests that adequate oxygen delivery may be restored at lower overall Hb concentration as the hct 15% level was able to perform efficiently like unmixed blood.

### RSR-13 Affects NO Binding to HBOC and Hb

Attempts to prevent or reduce HBOC-induced hypertension have focused on means to increase NO bioavailability by limiting NO-heme pocket interactions^{51, 52} or using pharmacologic methods of NO supplementation, such as through NO inhalation.^{81, 82} In a recent study with Oxyglobin® (polymerized bovine Hb), it was shown that pulmonary arterial pressure can be attenuated when the HBOC is administered with NO via S-nitrosylation.⁸³ Due to Hb secondary function as a nitrite reductase,⁶¹ nitrite administration has also been proposed as another option to balance the NO scavenging effect of HBOCs.^{54, 55, 84}

This study reports the potential use of RSR-13 to allosterically or otherwise reduce NO binding to Hb, and the first such study using an allosteric effector to prevent scavenging of NO by HBOC. In the absence of RSR-13, NOHb or NOHBOC-1 displays a high-affinity state, while addition of RSR-13 resulted in a low-affinity state, indicative of low affinity of NO binding to Hb in the presence of RSR-13. The effect of RSR-13 on the absorbance spectroscopic properties of nitrosylated Hb is in keeping with the observed functional behavior with oxygen. The observation that HBOCs and cell free Hb show decreased binding with NO support the therapeutic use of RSR-13 to mitigate NO binding by HBOCs.

### RSR-13 Reduces COHb Levels in Swine Blood and HBOC-1

HBOC-induced vasoconstriction as a result of scavenging of endogenous CO by extracellular Hb has been well documented.^{40, 69} Exogenously delivered CO has been shown to provide beneficial anti-inflammatory and tissue protective effects, and HBOC has been a proposed vehicle for delivering the CO. However, due to potential scavenging of CO by the HBOC or Hb, large doses of CO would have to be given to reach therapeutic level. This could result in significant toxicity problem, such as the risk of formation of COHb that would impair Hb function of oxygen delivery to tissues and organs. There are studies underway with CO-releasing molecules (CORMs) to deliver constant amounts of CO to tissues without significant impact on COHb levels.⁷⁰ Nonetheless, CORMs would not solve the problem associated with HBOC use or the potential CO toxicity problems associated with CORMs. Rather, a system when used in conjunction with HBOC to prevent or decrease binding of CO to HBOC would most be appropriate. To prove our hypothesis that RSR-13 would prevent or decrease CO binding to Hb, and thus can be used in conjunction with HBOC to deliver low-dose of CO and/or ameliorate the scavenging effect of HBOC, we studied the effect of RSR-13 on COHb level in swine blood and HBOC-1. The result shows that RSR-13 is able to decrease COHb levels in both HBOC-1 and swine blood. This study has clinical implication regarding the use of RSR-13 as an adjunct with HBOC to deliver small amounts of CO without compromising the Hb function to deliver oxygen. This would also prove to be valuable in the treatment of CO poisoning beyond that previously demonstrated by RSR-13 since the current invention demonstrates that RSR-13 is effective in decreasing CO binding to HBOCs which could be used to treat CO poisoning by delivery more oxygen and decreasing the poisoning of the HBOC with CO.

### SUMMARY

Exogenous gases, such as CO and NO have been shown to have therapeutic value, including anti-inflammation, vasodilation or tissue protection. Key to their effectiveness is enhancing their bioavailability in plasma so that they are free to interact with the vasculature and with the organ and immune cells of the body to exert their beneficial effects. However, there are several impediments to their use as a result of scavenging by Hb or HBOC's. For, example, due to its NO scavenging, use of HBOCs could be very dangerous, especially in the setting of hemorrhage, by raising hydrostatic pressure and thus causing additional hemorrhage. Furthermore, such NO scavenging may reduce blood flow to critical organ tissue in the setting of ischemia. CO has historically been perceived as a lethal gas, however, over the last 2 decades it has been shown that CO, similar to NO has therapeutic value. However, if used in larger amounts especially in cases of global hypoperfusion, the resulting levels of carboxyhemoglobin could be dangerous. This would be made worse where supplemental oxygen is not available (i.e. battlefield). Thus, means to reduce binding of these gases to either native hemoglobin or the hemoglobin of HBOC are needed to optimize their bioavailability and to reduce their cytotoxic effects.

Previous studies with RSR-13 show this compound to bind to Hb and decrease the heme affinity for oxygen and CO. Our present studies also show that RSR-13 is capable of decreasing HBOC, as well as erythrocyte Hb or HBOC/Hb mixtures affinity for oxygen, NO or CO. From the forgoing, it's obvious that RSR-13 can be used to reduce binding of NO or CO or other therapeutic gases that may bind with erythrocyte, HBOCs, or other metalloproteins and chrompohores that have gas carrying potential. This would mitigate the vasoconstriction side effects of these Hbs or blood substitutes. Similarly, RSR-13 can be used in conjunction with HBOC's which are designed as tissue CO delivery vehicles for therapeutic use of CO or other non-oxygen therapeutic gases, such as H₂S, SO₂, etc. RSR-13 can also be used to enhance the bioavailability of CO and NO or other non-oxygen therapeutic gases, when they are either delivered via inhalation, intravenously (with or without a carrier), transdermally, mucosally, gastrointestinally, or via any other means.

All references (including articles, patents and patent applications) cited herein are hereby incorporated by referenced in entirety.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

### REFERENCES

1. Benesch R, Benesch R E. The effect of organic phosphates from the human erythrocyte on the allosteric properties of hemoglobin. Biochem Biophys Res Commun 1967;26:162-167.

2. Lalezari I, Lalezari P, Poyart C, Marden M, Kister J, Bohn B, Fermi G, Perutz M F. New effectors of human hemoglobin: structure and function. Biochemistry 1990;29:1515-1523.

3. Lalezari I, Rahbar S, Lalezari P, Fermi G, Perutz M F. LR16, a compound with potent effects on the oxygen affinity of hemoglobin, on blood cholesterol, and on low density lipoprotein. Proc Natl Acad Sci U S A 1988;85:6117-6121.

4. Abraham D J, Wireko F C, Randad R S, Poyart C, Kister J, Bohn B, Liard J F, Kunert M P. Allosteric modifiers of hemoglobin: 2-[4-[[(3,5-disubstituted anilino)carbonyl]methyl]phenoxy]-2-methylpropionic acid derivatives that lower the oxygen affinity of hemoglobin in red cell suspensions, in whole blood, and in vivo in rats. Biochemistry 1992;31:9141-9149.

5. Wireko F C, Kellogg G E, Abraham D J. Allosteric modifiers of hemoglobin. 2. Crystallographically determined binding sites and hydrophobic binding/interaction analysis of novel hemoglobin oxygen effectors. J Med Chem 1991;34:758-767.

6. Randad R S, Mahran M A, Mehanna A S, Abraham D J. Allosteric modifiers of hemoglobin. 1. Design, synthesis, testing, and structure-allosteric activity relationship of novel hemoglobin oxygen affinity decreasing agents. J Med Chem 1991;34:752-757.

7. Safo M K, Moure C M, Burnett J C, Joshi G S, Abraham D J. High-resolution crystal structure of deoxy hemoglobin complexed with a potent allosteric effector. Protein Sci 2001;10:951-957.

8. Watanabe T, Takeda T, Omiya S, Hikoso S, Yamaguchi O, Nakano Y, Higuchi Y, Nakai A, Abe Y, Aki-Jin Y, Taniike M, Mizote I, Matsumura Y, Shimizu T, Nishida K, Imai K, Hori M, Shirasawa T, Otsu K. Reduction in hemoglobin-oxygen affinity results in the improvement of exercise capacity in mice with chronic heart failure. J Am Coll Cardiol 2008;52:779-786.

9. Scott C, Suh J, Stea B, Nabid A, Hackman J. Improved survival, quality of life, and quality-adjusted survival in breast cancer patients treated with efaproxiral (Efaproxyn) plus whole-brain radiation therapy for brain metastases. Am J Clin Oncol 2007;30:580-587.

10. Kaal E C, Vecht C J. CNS complications of breast cancer: current and emerging treatment options. CNS Drugs 2007;21:559-579.

11. Stea B, Suh J H, Boyd A P, Cagnoni P J, Shaw E, REACH Study Group. Whole-brain radiotherapy with or without efaproxiral for the treatment of brain metastases: Determinants of response and its prognostic value for subsequent survival. Int J Radiat Oncol Biol Phys 2006;64:1023-1030.

12. Suh J H, Stea B, Nabid A, Kresl J J, Fortin A, Mercier J P, Senzer N, Chang E L, Boyd A P, Cagnoni P J, Shaw E. Phase III study of efaproxiral as an adjunct to whole-brain radiation therapy for brain metastases. J Clin Oncol 2006;24:106-114.

13. Kunert M P, Liard J F, Abraham D J. RSR-13, an allosteric effector of hemoglobin, increases systemic and iliac vascular resistance in rats. Am J Physiol 1996;271:H602-13.

14. Kunert M P, Liard J F, Abraham D J, Lombard J H. Low-affinity hemoglobin increases tissue PO2 and decreases arteriolar diameter and flow in the rat cremaster muscle. Microvasc Res 1996;52:58-68.

15. Khandelwal S R, Randad R S, Lin P S, Meng H, Pittman R N, Kontos H A, Choi S C, Abraham D J, Schmidt-Ullrich R. Enhanced oxygenation in vivo by allosteric inhibitors of hemoglobin saturation. Am J Physiol 1993;265:H1450-3.

16. Grinberg O Y, Miyake M, Hou H, Steffen R P, Swartz H M. The dose-dependent effect of RSR13, a synthetic allosteric modifier of hemoglobin, on physiological parameters and brain tissue oxygenation in rats. Adv Exp Med Biol 2003;530:287-296.

17. Pagel P S, Hettrick D A, Montgomery M W, Kersten J R, Warltier D C. RSR13, a synthetic allosteric modifier of hemoglobin, enhances recovery of stunned myocardium in dogs. Adv Exp Med Biol 1998;454:527-531.

18. Stucker O, Laurent D, Duvelleroy M, Ropars C, Teisseire B. Incorporation of inositol hexaphosphate in stored erythrocytes: effect on tissue oxygenation. Life Support Syst 1985;3 Suppl 1:458-461.

19. Teisseire B, Ropars C, Villereal M C, Nicolau C. Long-term physiological effects of enhanced O2 release by inositol hexaphosphate-loaded erythrocytes. Proc Natl Acad Sci U S A 1987;84:6894-6898.

20. Villereal M C, Ropars C, Hurel C, Teisseire B, Chassaigne M, Itti R, Casset D, Nicolau C. Oxygen transport to tissue modified by entrapment of an allosteric effector of haemoglobin in erythrocytes. Folia Haematol Int Mag Klin Morphol Blutforsch 1987;114:488-492.

21. Perutz M F, Poyart C. Bezafibrate lowers oxygen affinity of haemoglobin. Lancet 1983;2:881-882.

22. Gong Q, Simplaceanu V, Lukin J A, Giovannelli J L, Ho N T, Ho C. Quaternary structure of carbonmonoxyhemoglobins in solution: structural changes induced by the allosteric effector inositol hexaphosphate. Biochemistry 2006;45:5140-5148.

23. Yonetani T, Tsuneshige A. The global allostery model of hemoglobin: an allosteric mechanism involving homotropic and heterotropic interactions. C R Biol 2003;326:523-532.

24. Safo M K, Boyiri T, Burnett J C, Danso-Danquah R, Moure C M, Joshi G S, Abraham D J. X-ray crystallographic analyses of symmetrical allosteric effectors of hemoglobin: compounds designed to link primary and secondary binding sites. Acta Crystallogr D Biol Crystallogr 2002;58:634-644.

25. Shibayama N, Miura S, Tame J R, Yonetani T, Park S Y. Crystal structure of horse carbonmonoxyhemoglobin-bezafibrate complex at 1.55-A resolution. A novel allosteric binding site in R-state hemoglobin. J Biol Chem 2002;277:38791-38796.

26. Schay G, Smeller L, Tsuneshige A, Yonetani T, Fidy J. Allosteric effectors influence the tetramer stability of both R- and T-states of hemoglobin A. J Biol Chem 2006;281:25972-25983.

27. Yokoyama T, Neya S, Tsuneshige A, Yonetani T, Park S Y, Tame J R. R-state haemoglobin with low oxygen affinity: crystal structures of deoxy human and carbonmonoxy horse haemoglobin bound to the effector molecule L35. J Mol Biol 2006;356:790-801.

28. Miyake M, Grinberg O Y, Hou H, Steffen R P, Elkadi H, Swartz H M. The effect of RSR13, a synthetic allosteric modifier of hemoglobin, on brain tissue pO2 (measured by EPR oximetry) following severe hemorrhagic shock in rats. Adv Exp Med Biol 2003;530:319-329.

29. Hou H, Khan N, O'Hara J A, Grinberg O Y, Dunn J F, Abajian M A, Wilmot C M, Demidenko E, Lu S, Steffen R P, Swartz H M. Increased oxygenation of intracranial tumors by efaproxyn (efaproxiral), an allosteric hemoglobin modifier: In vivo EPR oximetry study. Int J Radiat Oncol Biol Phys 2005;61:1503-1509.

30. Hou H, Khan N, O'Hara J A, Grinberg O Y, Dunn J F, Abajian M A, Wilmot C M, Makki M, Demidenko E, Lu S, Steffen R P, Swartz H M. Effect of RSR13, an allosteric hemoglobin modifier, on oxygenation in murine tumors: an in vivo electron paramagnetic resonance oximetry and bold MRI study. Int J Radiat Oncol Biol Phys 2004;59:834-843.

31. Hou H, Khan N, Grinberg O Y, Yu H, Grinberg S A, Lu S, Demidenko E, Steffen R P, Swartz H M. The effects of Efaproxyn (efaproxiral) on subcutaneous RIF-1 tumor oxygenation and enhancement of radiotherapy-mediated inhibition of tumor growth in mice. Radiat Res 2007;168:218-225.

32. Phelps Grella M, Danso-Danquah R, Safo M K, Joshi G S, Kister J, Marden M, Hoffman S J, Abraham D J. Synthesis and structure-activity relationships of chiral allosteric modifiers of hemoglobin. J Med Chem 2000;43:4726-4737.

33. Youssef A M, Safo M K, Danso-Danquah R, Joshi G S, Kister J, Marden M C, Abraham D J. Synthesis and X-ray studies of chiral allosteric modifiers of hemoglobin. J Med Chem 2002;45:1184-1195.

34. Kavanaugh J S, Rogers P H, Arnone A. Crystallographic evidence for a new ensemble of ligand-induced allosteric transitions in hemoglobin: the T-to-T(high) quaternary transitions. Biochemistry 2005;44:6101-6121.

35. Kavanaugh J S, Weydert J A, Rogers P H, Arnone A, Hui H L, Wierzba A M, Kwiatkowski L D, Paily P, Noble R W, Bruno S, Mozzarelli A. Site-directed mutations of human hemoglobin at residue 35beta: a residue at the intersection of the alpha1beta1, alpha1beta2, and alpha1alpha2 interfaces. Protein Sci 2001;10:1847-1855.

36. Chen Q, Lalezari I, Nagel R L, Hirsch R E. Liganded hemoglobin structural perturbations by the allosteric effector L35. Biophys J 2005;88:2057-2067.

37. Safo M K, Ahmed M H, Ghatge M S, Boyiri T. Hemoglobin-ligand binding: Understanding Hb function and allostery on atomic level. Biochim Biophys Acta 2011;1814:797-809.

38. Cosby K, Partovi K S, Crawford J H, Patel R P, Reiter C D, Martyr S, Yang B K, Waclawiw M A, Zalos G, Xu X, Huang K T, Shields H, Kim-Shapiro D B, Schechter A N, Cannon RO,3rd, Gladwin M T. Nitrite reduction to nitric oxide by deoxyhemoglobin vasodilates the human circulation. Nat Med 2003;9:1498-1505.

39. Wu L, Wang R. Carbon monoxide: endogenous production, physiological functions, and pharmacological applications. Pharmacol Rev 2005;57:585-630.

40. Goda N, Suzuki K, Naito M, Takeoka S, Tsuchida E, Ishimura Y, Tamatani T, Suematsu M. Distribution of heme oxygenase isoforms in rat liver. Topographic basis for carbon monoxide-mediated microvascular relaxation. J Clin Invest 1998;101:604-612.

41. Suematsu M, Goda N, Sano T, Kashiwagi S, Egawa T, Shinoda Y, Ishimura Y. Carbon monoxide: an endogenous modulator of sinusoidal tone in the perfused rat liver. J Clin Invest 1995;96:2431-2437.

42. Suematsu M, Kashiwagi S, Sano T, Goda N, Shinoda Y, Ishimura Y. Carbon monoxide as an endogenous modulator of hepatic vascular perfusion. Biochem Biophys Res Commun 1994;205:1333-1337.

43. Maley J H, Lasker G F, Kadowitz P J. Nitric oxide and disorders of the erythrocyte: emerging roles and therapeutic targets. Cardiovasc Hematol Disord Drug Targets 2010;10:284-291.

44. Rossaint R, Falke K J, Lopez F, Slama K, Pison U, Zapol W M. Inhaled nitric oxide for the adult respiratory distress syndrome. N Engl J Med 1993;328:399-405.

45. Pison U, Lopez F A, Heidelmeyer C F, Rossaint R, Falke K J. Inhaled nitric oxide reverses hypoxic pulmonary vasoconstriction without impairing gas exchange. J Appl Physiol 1993;74:1287-1292.

46. Roberts J D,Jr, Fineman J R, Morin F C,3rd, Shaul P W, Rimar S, Schreiber M D, Polin R A, Zwass M S, Zayek M M, Gross I, Heymann M A, Zapol W M. Inhaled nitric oxide and persistent pulmonary hypertension of the newborn. The Inhaled Nitric Oxide Study Group. N Engl J Med 1997;336:605-610.

47. Bloch K D, Ichinose F, Roberts J D,Jr, Zapol W M. Inhaled NO as a therapeutic agent. Cardiovasc Res 2007;75:339-348.

48. Ichinose F, Roberts J D,Jr, Zapol W M. Inhaled nitric oxide: a selective pulmonary vasodilator: current uses and therapeutic potential. Circulation 2004;109:3106-3111.

49. Roberts J D,Jr, Chiche J D, Weimann J, Steudel W, Zapol W M, Bloch K D. Nitric oxide inhalation decreases pulmonary artery remodeling in the injured lungs of rat pups. Circ Res 2000;87:140-145.

50. Bonaventura C, Henkens R, Alayash A I, Crumbliss A L. Allosteric effects on oxidative and nitrosative reactions of cell-free hemoglobins. IUBMB Life 2007;59:498-505.

51. Olson J S, Foley E W, Rogge C, Tsai A L, Doyle M P, Lemon D D. No scavenging and the hypertensive effect of hemoglobin-based blood substitutes. Free Radic Biol Med 2004;36:685-697.

52. Doherty D H, Doyle M P, Curry S R, Vali R J, Fattor T J, Olson J S, Lemon D D. Rate of reaction with nitric oxide determines the hypertensive effect of cell-free hemoglobin. Nat Biotechnol 1998;16:672-676.

53. Rother R P, Bell L, Hillmen P, Gladwin M T. The clinical sequelae of intravascular hemolysis and extracellular plasma hemoglobin: a novel mechanism of human disease. JAMA 2005;293:1653-1662.

54. Silverman T A, Weiskopf R B. Hemoglobin-based oxygen carriers: current status and future directions. Transfusion 2009;49:2495-2515.

55. Silverman T A, Weiskopf R B, Planning Committee and the Speakers. Hemoglobin-based oxygen carriers: current status and future directions. Anesthesiology 2009;111:946-963.

56. Weiskopf R B. Hemoglobin-based oxygen carriers: compassionate use and compassionate clinical trials. Anesth Analg 2010;110:659-662.

57. Furchgott R F. Endothelium-derived relaxing factor: discovery, early studies, and identification as nitric oxide. Biosci Rep 1999;19:235-251.

58. Furchgott R F, Jothianandan D. Endothelium-dependent and -independent vasodilation involving cyclic GMP: relaxation induced by nitric oxide, carbon monoxide and light. Blood Vessels 1991;28:52-61.

59. Furchgott R F, Vanhoutte P M. Endothelium-derived relaxing and contracting factors. FASEB J 1989;3:2007-2018.

60. Furchgott R F, Zawadzki J V. The obligatory role of endothelial cells in the relaxation of arterial smooth muscle by acetylcholine. Nature 1980;288:373-376.

61. Gladwin M T, Schechter A N, Kim-Shapiro D B, Patel R P, Hogg N, Shiva S, Cannon R O,3rd, Kelm M, Wink D A, Espey M G, Oldfield E H, Pluta R M, Freeman B A, Lancaster J R,Jr, Feelisch M, Lundberg J O. The emerging biology of the nitrite anion. Nat Chem Biol 2005;1:308-314.

62. Hess J R, MacDonald V W, Brinkley W W. Systemic and pulmonary hypertension after resuscitation with cell-free hemoglobin. J Appl Physiol 1993;74:1769-1778.

63. Caumartin Y, Stephen J, Deng J P, Lian D, Lan Z, Liu W, Garcia B, Jevnikar A M, Wang H, Cepinskas G, Luke P P. Carbon monoxide-releasing molecules protect against ischemia-reperfusion injury during kidney transplantation. Kidney Int 2011;79:1080-1089.

64. Nakao A, Choi A M, Murase N. Protective effect of carbon monoxide in transplantation. J Cell Mol Med 2006;10:650-671.

65. Nakao A, Toyokawa H, Abe M, Kiyomoto T, Nakahira K, Choi A M, Nalesnik M A, Thomson A W, Murase N. Heart allograft protection with low-dose carbon monoxide inhalation: effects on inflammatory mediators and alloreactive T-cell responses. Transplantation 2006;81:220-230.

66. Kohmoto J, Nakao A, Kaizu T, Tsung A, Ikeda A, Tomiyama K, Billiar T R, Choi A M, Murase N, McCurry K R. Low-dose carbon monoxide inhalation prevents ischemia/reperfusion injury of transplanted rat lung grafts. Surgery 2006;140:179-185.

67. Nakao A. Kimizuka K, Stolz D B, Seda Neto J, Kaizu T, Choi A M, Uchiyama T, Zuckerbraun B S, Bauer A J, Nalesnik M A, Otterbein L E, Geller D A, Murase N. Protective effect of carbon monoxide inhalation for cold-preserved small intestinal grafts. Surgery 2003;134:285-292.

68. Kaizu T, Nakao A, Tsung A, Toyokawa H, Sahai R, Geller D A, Murase N. Carbon monoxide inhalation ameliorates cold ischemia/reperfusion injury after rat liver transplantation. Surgery 2005;138:229-235.

69. Sakai H, Okuda N, Sato A, Yamaue T, Takeoka S, Tsuchida E. Hemoglobin encapsulation in vesicles retards NO and CO binding and O2 release when perfused through narrow gas-permeable tubes. Am J Physiol Heart Circ Physiol 2010;298:H956-65.

70. Motterlini R, Mann B E, Johnson T R, Clark J E, Foresti R, Green C J. Bioactivity and pharmacological actions of carbon monoxide-releasing molecules. Curr Pharm Des 2003;9:2525-2539.

71. Hart J L. Role of sulfur-containing gaseous substances in the cardiovascular system. Front Biosci (Elite Ed) 2011;3:736-749.

72. Wang X B, Jin H F, Tang C S, Du J B. Significance of endogenous sulphur-containing gases in the cardiovascular system. Clin Exp Pharmacol Physiol 2010;37:745-752.

73. Liang Y, Liu D, Ochs T, Tang C, Chen S, Zhang S, Geng B, Jin H, Du J. Endogenous sulfur dioxide protects against isoproterenol-induced myocardial injury and increases myocardial antioxidant capacity in rats. Lab Invest 2011;91:12-23.

74. Liu D, Jin H, Tang C, Du J. Sulfur dioxide: a novel gaseous signal in the regulation of cardiovascular functions. Mini Rev Med Chem 2010;10:1039-1045.

75. Sun Y, Tian Y, Prabha M, Liu D, Chen S, Zhang R, Liu X, Tang C, Tang X, Jin H, Du J. Effects of sulfur dioxide on hypoxic pulmonary vascular structural remodeling. Lab Invest 2010;90:68-82.

76. Schedin U, Frostell C G, Gustafsson L E. Formation of nitrogen dioxide from nitric oxide and their measurement in clinically relevant circumstances. Br J Anaesth 1999;82:182-192.

77. Szabo C. Hydrogen sulphide and its therapeutic potential. Nat Rev Drug Discov 2007;6:917-935.

78. Thatcher G R, Nicolescu A C, Bennett B M, Toader V. Nitrates and NO release: contemporary aspects in biological and medicinal chemistry. Free Radic Biol Med 2004;37:1122-1143.

79. Safo M K, Abraham D J. X-ray crystallography of hemoglobins. Methods Mol Med 2003;82:1-19.

80. Ascenzi P, Coletta M, Desideri A, Polizio F, Bertollini A, Santucci R, Amiconi G. Effect of bezafibrate and clofibric acid on the spectroscopic properties of the nitric oxide derivative of ferrous human hemoglobin. J Inorg Biochem 1992;48:47-53.

81. Yu B, Raher M J, Volpato G P, Bloch K D, Ichinose F, Zapol W M. Inhaled nitric oxide enables artificial blood transfusion without hypertension. Circulation 2008;117:1982-1990.

82. Yu B, Bloch K D, Zapol W M. Hemoglobin-based red blood cell substitutes and nitric oxide. Trends Cardiovasc Med 2009;19:103-107.

83. Irwin D, Buehler P W, Alayash A I, Jia Y, Bonventura J. Foreman B, White M, Jacobs R, Piteo B, TissotvanPatot M C, Hamilton K L, Gotshall R W. Mixed S-nitrosylated polymerized bovine hemoglobin species moderate hemodynamic effects in acutely hypoxic rats. Am J Respir Cell Mol Biol 2010;42:200-209.

84. Gladwin M T, Kim-Shapiro D B. The functional nitrite reductase activity of the hemeglobins. Blood 2008;112:2636-2647.

1. A method of providing at least one medicinal gas to a subject in need thereof, wherein said at least one medicinal gas is selected from the group consisting of NO, H₂S, N₂O, SO, SO₂ and O₂, comprising,
administering to said subject an agent that changes the binding of said at least one medicinal gas to hemoglobin (Hb); and
providing to said subject either
i) said at least one medicinal gas, or
ii) at least one source of said at least one medicinal gas, with the caveat that if said at least one medicinal gas is O₂, then at least one additional medicinal gas is also administered.

2. The method of claim 1, wherein said step of providing is carried out by exogenous administration.

3. The method of claim 1, wherein said step of providing includes a step of applying to said subject at least one exogenous stimulus which elicits endogenous production of said at least one medicinal gas.

4. (canceled)

5. The method of claim 1, wherein said at least one medicinal gas is not O₂.

6. The method of claim 1, further comprising the step of administering one or both of a perfluorocarbon (PFC) and at least one hemoglobin-based oxygen carrier (HBOC) to said subject.

7. The method of claim 6, wherein said step of providing said at least one medicinal gas is carried out by inhalation and said PFC is administered prior to said step of providing.

8. The method of claim 1, wherein said at least one source of said at least one medicinal gas is a PFC that comprises said at least one medicinal gas.

9. The method of claim 8, wherein said PFC that comprises said at least one medicinal gas is administered intravenously.

10. The method of claim 1, wherein said that changes the binding of the at least one medicinal gas to Hb is an allosteric modulator or effector of Hb.

11. The method of claim 10, wherein said allosteric modulator or effector of Hb is a compound of Formula I 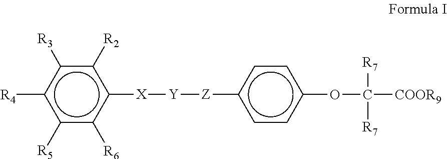
wherein X, Y and Z are independently selected from CH₂, NH, or O; R₂₋₆ are independently selected from hydrogen, halogen, a substituted or unsubstituted C₁, C₂, or C₃ alkyl group, and alkyl moieties of aliphatic or aromatic rings incorporating two of the R₂₋₆ sites; R₇₋₈ are independently selected from hydrogen, methyl, ethyl groups and alkyl moieties as part of an aliphatic ring connecting R₇ and R₈; and R₉ is hydrogen, methyl, ethyl, propyl, or a salt cation.

12. The method of claim 11, wherein said salt cation is sodium, potassium, or ammonium.

13. The method of claim 11, wherein said allosteric modulator or effector of Hb is

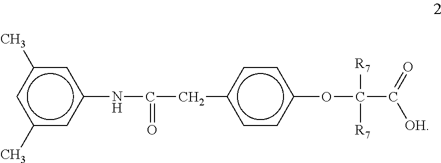

14. The method of claim 10, wherein said allosteric modulator or effector of Hb is 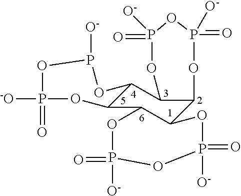
or a functional variant, analog, isomer or salt thereof.

15. A method of delivering oxygen (O₂) to cells and tissues of a patient in need thereof, comprising the steps of
co-administering to said patient
1) at least one hemoglobin-based oxygen carrier (HBOC); and
2) an agent that changes the binding of O₂ to said at least one HBOC.

16-24. (canceled)

25. A method of changing the binding of O₂ to a hemoglobin based oxygen carrier (HBOC), comprising the step of
exposing said HBOC to an agent that increases the P₅₀ of said Hb.

26-27. (canceled)

28. A method of diagnosing a condition or illness characterized by increased production of one or more of endogenous CO, H₂S and NO in a patient in need thereof, comprising the steps of
administering to said patient an agent that changes the binding of one or more of CO, H₂S and NO to Hb;
obtaining a biological sample form said patient;
detecting a level of one or more of NO, CO, H₂S, N₂O, SO, SO₂ and O₂ in said biological sample; and:
i) if said level of one or more of NO, CO, H₂S, N₂O, SO, SO₂ and O₂ in said biological sample is higher than a predetermined control level, then concluding that said patient has said condition or said illness; and
ii) if said level of one or both of NO, CO, H₂S, N₂O, SO, SO₂ and O₂ in said biological sample is not higher than said predetermined control level, then concluding that said patient does not have said condition or said illness.

29.

30. A method of treating gas poisoning in a patient in need thereof, comprising the step of
co-administering to said patient
i) an agent that changes the binding of said gas to hemoglobin; and
ii) one or both of a hemoglobin based oxygen carrier (HBOC) and a perfluorocarbon (PFC).

31. (canceled)

32. The method of claim 1, wherein said subject is a non-human animal.

33. The method of claim 15, wherein said patient is a non-human animal.

34. The method of claim 28, wherein said patient is a non-human animal.

35. The method of claim 30, wherein said patient is a non-human animal.

36. The method of claim 1 wherein the subject has tissue hypoxia and/or inflammation due to hemorrhagic shock.

37. The method of claim 36, wherein said allosteric modulator or effector of Hb is [chemical structure]
